(19) United States
(12) Patent Application Publication
(10) Pub. No.: US 2025/0367261 A1
(43) Pub. Date: Dec. 4, 2025

(54) GOSERELIN IMPLANT DOSING REGIMENS FOR IMPROVED PATIENT COMPLIANCE

(71) Applicant: TerSera Therapeutics LLC, Deerfield, IL (US)

(72) Inventors: Nancy Elizabeth Martin, Deerfield, IL (US); Lonnie Don Brent, Deerfield, IL (US)

(73) Assignee: TerSera Therapeutics LLC, Deerfield, IL (US)

(21) Appl. No.: 18/733,464

(22) Filed: Jun. 4, 2024

(51) Int. Cl. A61K 38/24; A61K 45/06

(52) U.S. Cl. CPC A61K 38/24; A61K 45/06

(57) ABSTRACT

Described herein are methods of treating hormone receptor positive breast cancer in a patient which include concomitantly administering an initial neoadjuvant or adjuvant IV chemotherapy cycle and an initial dose of a 3.6 mg goserelin acetate implant to the patient, or concomitantly administering an initial dose of a 3.6 mg goserelin acetate implant to the patient with a first of a plurality of long-term IV chemotherapy cycles. After one or more doses of the 3.6 mg goserelin acetate implant, the 3.6 mg goserelin acetate implant is discontinued, and the patient is then administered a 10.8 mg goserelin implant once every 84 to 108 days. Also included are methods of improving patient compliance, increasing duration of treatment, and/or reducing the need for oophorectomy in a patient with hormone receptor positive breast cancer by administering a 3.6 mg goserelin acetate implant to the patient, and upon the patient becoming nonadherent, the 3.6 mg goserelin acetate implant is discontinued, and the patient is then administered a 10.8 mg goserelin implant once every 84 to 108 days.

### FIELD OF THE DISCLOSURE

The present disclosure is related to dosing regimens for goserelin implant dosage forms that can improve patient safety and compliance.

### BACKGROUND

Goserelin is a gonadotropin-releasing hormone (GnRH) agonist used, for example, in ovarian function suppression in the treatment of pre- and peri-menopausal patients with advanced breast cancer. Goserelin, administered as a subcutaneous implant, is available in doses of 3.6 mg for once monthly administration and 10.8 mg for administration once every three months. The goserelin 3.6 mg implant (Zoladex®) is approved in the United States for the palliative treatment of advanced breast cancer, while the 10.8 mg implant is currently not approved for this indication. Both the 3.6 mg and 10.8 mg doses are approved by Health Canada and the European Medicines Agency for the treatment of breast cancer.

Described herein are dosage regimens for the treatment of breast cancer patients, particularly regimens that can improve patient compliance.

### BRIEF SUMMARY

In an aspect, a method of treating hormone receptor positive breast cancer in a patient comprises
concomitantly administering an initial neoadjuvant or adjuvant IV chemotherapy cycle and an initial dose of a 3.6 mg goserelin acetate implant to the patient, optionally administering subsequent neoadjuvant or adjuvant IV chemotherapy cycles to the patient, wherein the subsequent neoadjuvant or adjuvant IV chemotherapy cycles are the same or different from the initial neoadjuvant or adjuvant IV chemotherapy cycle and each other, and either
   i) 28-36 days after the initial dose of the 3.6 mg goserelin acetate implant, administering a second dose of the 3.6 mg goserelin acetate implant to the patient,
   optionally administering subsequent doses of the 3.6 mg goserelin acetate implant to the patient every 28-36 days, and
   after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days,
   or
   ii) after the initial dose of the 3.6 mg goserelin acetate implant, discontinuing the 3.6 mg goserelin acetate implant, and administering a 10.8 mg goserelin implant to the patient once every 84 to 108 days.

In another aspect, a method of treating hormone receptor positive breast cancer in a patient comprises
administering a plurality of long-term IV chemotherapy cycles to the patient to provide a long-term course of chemotherapy,
concomitantly administering an initial dose of a 3.6 mg goserelin acetate implant to the patient when a first of the plurality of long-term IV chemotherapy cycles is administered, and either
   i) 28-36 days after the initial dose of the 3.6 mg goserelin acetate implant, administering a second dose of the 3.6 mg goserelin acetate implant to the patient,
   optionally administering subsequent doses of the 3.6 mg goserelin acetate implant to the patient every 28-36 days, and
   after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days,
   or
   ii) after the initial dose of the 3.6 mg goserelin acetate implant, discontinuing the 3.6 mg goserelin acetate implant, and administering a 10.8 mg goserelin implant to the patient once every 84 to 108 days,
wherein in i) or ii) administering the 10.8 mg goserelin implant to the patient once every 84 to 108 days is continued for the lifetime of the patient.

In a further aspect, a method of improving patient compliance, increasing duration of treatment, and/or reducing the need for oophorectomy in a patient with hormone receptor positive breast cancer comprises
i) administering an initial dose of a 3.6 mg goserelin acetate implant to the patient,
administering one or more subsequent doses of the 3.6 mg goserelin acetate implant to the patient, wherein each subsequent dose is prescribed to be administered 4 weeks after a preceding dose,
identifying the patient as nonadherent if the patient does not receive the subsequent dose of 3.6 mg goserelin acetate implant at less than or equal to 36 days after the preceding dose,
discontinuing the 3.6 mg goserelin acetate implant in the nonadherent patient,
administering a 10.8 mg goserelin implant to the nonadherent patient, wherein the 10.8 mg goserelin implant is prescribed to be administered once every 12 weeks, and
continuing to administer the 10.8 mg goserelin implant is administered once every 84 to 108 days to improve patient compliance, increase duration of treatment, and/or reduce the need for oophorectomy,
wherein a course of treatment with a combination of 3.6 mg goserelin acetate implant and 10.8 mg goserelin implant is two years or the lifetime of the patient;
or
ii) administering an initial dose of a 3.6 mg goserelin acetate implant to the patient, wherein a subsequent dose is prescribed to be administered 4 weeks after the first dose,
identifying the patient as nonadherent when the patient does not receive the subsequent dose of 3.6 mg goserelin acetate implant at less than or equal to 36 days after the first dose,
discontinuing the 3.6 mg goserelin acetate implant in the nonadherent patient, and
administering a 10.8 mg goserelin implant to the nonadherent patient, wherein the 10.8 mg goserelin implant is prescribed to be administered once every 12 weeks,
wherein the 10.8 mg goserelin implant is administered once every 84 to 108 days for a minimum of eight doses to improve patient compliance, increase duration of treatment and/or reduce the need for oophorectomy.

### DETAILED DESCRIPTION

Described herein are dosing regimens for goserelin implants based on a study utilizing US real-world electronic health record evidence to characterize treatment patterns of patients treated with goserelin 3.6 mg and/or 10.8 mg. While the goserelin 10.8 mg dose is not currently approved for the treatment of breast cancer, it is sometimes prescribed off-label for the treatment of breast cancer. Upon review of the study data, the inventors recognized the existing dosages of 3.6 mg once per month and 10.8 mg once every 3 months could be leveraged into dosing regimens that improve both patient safety and compliance.

Goserelin is described in U.S. Pat. No. 4,100,274. Goserelin acetate has the chemical formula pyro-Glu His Trp Ser Tyr D-Ser(Bu^{t})Leu Arg Pro Azgly NH₂ acetate.

The controlled release depot formulation of goserelin acetate is described in U.S. Pat. No. 4,767,628. The current commercial Zoladex® products are cylindrical solid forms containing goserelin acetate in a matrix of D,L-lactic and glycolic acids copolymer preloaded in a special syringe for subcutaneous administration. U.S. Pat. No. 5,366,734 describes a method of continuous release from an implantable/injectable composition.

Described herein are regimens for administering goserelin 3.6 mg and 10.8 mg to breast cancer patients including regimens for administering both chemotherapy and goserelin 3.6 mg and 10.8 mg to breast cancer patients.

The stages of breast cancer are generally defined as follows:
0: no evidence of primary breast tumor, includes ductal carcinoma in situ
IA: breast tumor 2 cm or less across, no cancer in lymph nodes
IB: no breast tumor or breast tumor 2 cm or less across, cancer in lymph nodes
IIA: no breast tumor or breast tumor 2 cm or less, with cancer in lymph nodes under the arm; or breast tumor 2 cm to not more than 5 cm across, cancer in lymph nodes
IIB: breast tumor 2 cm to not more than 5 cm across, cancer in one to three lymph nodes; or breast tumor larger than 5 cm, no cancer in lymph nodes
IIIA: with or without breast tumor, cancer in four to nine lymph nodes; or breast tumor larger than 5 cm, cancer in one to three lymph nodes
IIIB: tumor has spread to the chest wall behind the breast, cancer may have spread to the skin, broken the skin, or spread to auxiliary lymph nodes
IIIC: tumor of any size in the breast, cancer has spread to one or more auxiliary lymph nodes, lymph nodes near the collarbone, underarm lymph nodes and lymph nodes near the breastbone, the skin
IV: cancer has spread to lymph nodes and distant part of the body beyond the breast, cancer metastasized to bones, lungs, brain or liver, for example

Stage IA and IB breast cancers are typically treated with surgery to remove the primary tumor and/or lymph nodes followed by a course of radiation therapy, and optionally hormone therapy for hormone-positive tumors and/or targeted cancer therapy. In some instances, adjuvant chemotherapy is used.

Stage IIA and IIB breast cancers are typically treated with surgery to remove the primary tumor or total mastectomy accompanied by removal of lymph nodes. Neoadjuvant and/or adjuvant chemotherapy is typically used. A course of radiation therapy, and optionally hormone therapy for hormone-positive tumors, targeted cancer therapy, and/or immunotherapy can also be used.

Stage 0, stage I and stage II breast cancer can collectively be referred to as early-stage breast cancer.

Stage IIIA, IIIB and IIIC breast cancers are typically treated with surgery, typically a total mastectomy and lymph node removal, accompanied by neoadjuvant and/or adjuvant chemotherapy. Radiation therapy, hormone therapy for hormone-positive tumors, targeted cancer therapy, and/or immunotherapy may also be used.

Stage IV breast cancer is primarily treated with chemotherapy, optionally with local and regional treatments such as surgery, radiation, or regional chemotherapy. Hormone therapy for hormone-positive tumors, targeted cancer therapy, and/or immunotherapy may also be used. Once a chemotherapy regimen has been identified, stage IV breast cancers can be treated with maintenance chemotherapy.

Stage III and stage IV breast cancer are advanced breast cancers. As used herein, advanced breast cancer includes locally advanced breast cancer as well as metastatic breast cancer.

In the United States, Zoladex® is approved for the treatment of advanced breast cancer, however, the methods described herein can also be used to treat early-stage breast cancer.

Chemotherapy is broadly defined as treatment to stop the growth of cancer cells, either by killing them or by preventing cancer cells from dividing. In general, chemotherapies target fast growing cells because cancer cells grow and divide faster than most cells in the body.

As used herein, the term chemotherapy includes neoadjuvant chemotherapy, adjuvant chemotherapy, and long-term chemotherapy.

Neoadjuvant chemotherapy is administered prior to surgery to shrink tumors or stop the spread of cancer. Adjuvant chemotherapy is administered after surgery to kill remaining cancer cells and reduce the chance of cancer recurrence. Neoadjuvant and adjuvant chemotherapy are typically administered over a course of 1, 2, 3, 4, 5, 6, 7, 8, 9, 10, 11, 12 or more cycles over a course of up to 12 months, for example.

Long-term chemotherapy includes maintenance chemotherapy and chemotherapy administered in response to reactivation of cancer. Maintenance chemotherapy refers to chemotherapy given on a regular schedule to help curb the spread of the cancer and prolong survival. Chemotherapy administered in response to reactivation of cancer can be administered, for example, when imaging tests and/or blood tests indicate reactivation of cancer, such as after surgery and termination of an initial round of adjuvant chemotherapy. Long-term chemotherapy includes chemotherapy administered over the lifetime of the patient.

Intravenous chemotherapy (IV chemotherapy) is given as an injection or a drip into a vein. IV chemotherapy can be administered in a doctor's office, infusion center, or hospital. Exemplary IV chemotherapies used for the treatment of breast cancer include 5-fluorouracil, capecitabine, carboplatin, cyclophosphamide, docetaxel, doxorubicin, daunorubicin, epirubicin, idarubicin, mitoxantrone, valrubicin, methotrexate, paclitaxel, ixabepilone, eribulin, vinorelbine, gemcitabine, capecitabine, floxuridine, cytarabine, decitabine, vidaza, and the like.

Chemotherapy treatments, particularly IV chemotherapy treatments, are typically administered in repeating cycles typically ranging from 1, 2, 3, 4, 5, 6, 7, or 8 week cycles. The break between treatments allows the body to rest and gain strength prior to the next cycle.

A series of chemotherapy cycles is referred to as a course of treatment, which can be 1 to 12 cycles (neoadjuvant or adjuvant chemotherapy) or longer (long-term chemotherapy). In the case of neoadjuvant or adjuvant chemotherapy, a course of chemotherapy may be administered up to 12 months, for example, such as 8 months to 12 months. In the case of long-term chemotherapy, a course of chemotherapy may last the lifetime of the patient.

As used herein, an initial IV chemotherapy cycle is the first cycle administered to the patient prior to surgery in the case of neoadjuvant therapy, the first cycle initiated after surgery in the case of adjuvant therapy, or the first cycle of long-term chemotherapy administered to the patient. In some of the methods described herein, it is advantageous to begin treatment with a 3.6 mg goserelin implant substantially when chemotherapy is started. Without being held to theory it is believed that concomitant administration of goserelin implant and chemotherapy can provide ovarian protection as well as support a reduction in the recurrence of cancer.

Subsequent IV chemotherapy cycles are the same or different from the initial chemotherapy cycle and each other, meaning the agents administered and/or the time between administrations may be the same or different.

As used herein, the term concomitant means that two therapies are administered during a specified time-period. As used herein, the specified time-period can be 2 weeks or shorter, such as 1 week, or 1 day such as in the same clinic visit.

In addition to chemotherapy, additional cancer therapies such as radiation therapy, hormone therapy, targeted cancer therapy, and/or immunotherapy can be administered to the patient.

Radiation therapy uses high energy X-rays, protons or other particles to kill cancer cells. External beam radiation therapy (EBRT) is the most common type of radiation therapy used in the treatment of breast cancer. Whole breast irradiation, accelerated partial breast irradiation, and chest wall irradiation can be used in the treatment of breast cancer post-surgery.

Hormone therapy can be administered before or after surgery to patients with estrogen-receptor positive (ER+) and/or progesterone positive (PR+) tumors). Exemplary hormone therapies include selective estrogen receptor modulators such as tamoxifen and toremifene (Fareston®); selective estrogen receptor degraders such as fulvestrant (Faslodex®) and elacestrant (Oserdu®); and aromatase inhibitors which lower estrogen levels such as letrozole (Femara®), anastrazole (Arimidex®), and exemestane (Aromasin®).

Targeted breast cancer therapies use drugs that block the activity of specific tumor targets such as HER2, mTor, P13K, and PARP. HER2 inhibitors include trastuzumab (Herceptin®), pertuzumab (Perjeta®), margetuximab-cmkb (Margenza™M), ado-trastuzumab emtansine (Kadcyla®), fam-trastuzumab deruxtecan (Enhertu®), lapatinib (Tykerb®), neratinib (Nerlynx®), and tucatinib (Tukysa®). mTor inhibitors include everolimus (Afinitor®). P13K inhibitors include alpelisib (Piqray®). PARP inhibitors include olaparib (Lynparza®), alaparib (Lynparza®), rucaparib (Rubraca®), talazoparib (Talzenna®), and niraparib (Zejula®). There is some overlap between targeted breast cancer therapies and immunotherapies, discussed below.

As used herein, immunotherapies are therapies that use a patient's own immune system to help kill cancer cells. Immunotherapies to treat breast cancer include margetuximab-cmkb (Margenza™): a monoclonal antibody that targets the HER2 pathway, pertuzumab (Perjeta®): a monoclonal antibody that targets the HER2 pathway, sacituzumab govitecan (Trodelvy®): an antibody-drug conjugate that targets the TROP-2 pathway, trastuzumab (Herceptin®): a monoclonal antibody that targets the HER2 pathway, trastuzumab deruxtecan (Enhertu®): an antibody-drug conjugate that targets the HER2 pathway and delivers toxic drugs to tumors, trastuzumab emtansine (Kadcyla®), PD-L1 inhibitor antibodies (atezolizumab (Tecentriq®), durvalumab (Imfinzi®), avelumab (Bavencio®)), PD-1 inhibitor antibodies (pembrolizumab (Keytruda®), nivolumab (Opdivo®), dostarlimab (Jemperli®), cemiplimab (Libtayo®)), CTLA-4 inhibitor antibodies (tremelimumab (Imjudo®), ipilimumab (Yervoy®), Quavonlimab, zalifrelimab), and combinations thereof.

As described herein, a method of treating hormone receptor positive breast cancer in a patient comprises concomitantly administering an initial IV chemotherapy cycle and an initial dose of a 3.6 mg goserelin acetate implant to the woman. When IV chemotherapy is initiated in a breast cancer patient, whether neoadjuvant, adjuvant or long-term therapy, the patient is typically visiting a doctor's office, infusion center, or hospital frequently, often once per week. Because Zoladex® is administered subcutaneously by a medical professional, it is advantageous to begin 3.6 mg Zoladex® concomitantly with the initiation of IV chemotherapy. Concomitant administration of chemotherapy and 3.6 mg Zoladex® can reduce the number of individual visits the patients must make, reducing stress on the patient. Also, concomitant administration of chemotherapy and 3.6 mg Zoladex® allows for careful monitoring tolerance to Zoladex® such as monitoring side effects caused by co-administration of chemotherapy and 3.6 mg Zoladex®. Common side effects of 3.6 mg Zoladex® include hot flashes, tumor flares, nausea, edema, malaise/fatigue/lethargy, and/or vomiting. Advantageously, concomitant administration of goserelin at the start of chemotherapy ensures careful monitoring and the beginning of therapy and adequate hormonal suppression and cessation of menses (a clinical indicator of adequate hormonal suppression).

As defined herein, a patient taking the 3.6 mg dose of a goserelin implant is considered to be compliant if a subsequent dose of the 3.6 mg goserelin acetate implant is administered 28-36 days after the previous dose, such as 28-36 days after the initial dose. A patient taking the 10.8 mg dose of a goserelin implant is considered to be compliant if a subsequent dose of the 10.8 mg goserelin acetate implant is administered 84 to 108 days after the previous dose, such as 84 to 108 days after the initial dose.

The methods described herein can be used to treat early-stage and advanced breast cancer. The methods can also be used to treat premenopausal, perimenopausal and menopausal women.

### Neoadjuvant or Adjuvant Chemotherapy

In an aspect, a method of treating hormone receptor positive breast cancer in a patient comprises concomitantly administering an initial neoadjuvant or adjuvant IV chemotherapy cycle and an initial dose of a 3.6 mg goserelin acetate implant to the patient, optionally administering subsequent neoadjuvant or adjuvant IV chemotherapy cycles to the patient, wherein the subsequent neoadjuvant or adjuvant IV chemotherapy cycles are the same or different from the initial neoadjuvant or adjuvant IV chemotherapy cycle and each other, and either:
i) 28-36 days after the initial dose of the 3.6 mg goserelin acetate implant, administering a second dose of the 3.6 mg goserelin acetate implant to the patient, optionally administering subsequent doses of the 3.6 mg goserelin acetate implant to the patient every 28-36 days, and after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days, or
ii) after the initial dose of the 3.6 mg goserelin acetate implant, discontinuing the 3.6 mg goserelin acetate implant, and administering a 10.8 mg goserelin implant to the patient once every 84 to 108 days.

In an aspect, 1-12 (1, 2, 3, 4, 5, 6, 7, 8, 9, 10, 11, 12) subsequent neoadjuvant or adjuvant IV chemotherapy cycles may be administered to the patient.

In aspect i), 28-36 days after the initial dose of the 3.6 mg goserelin acetate implant is administered, a second dose of the 3.6 mg goserelin acetate implant is administered to the patient. That is, the patient is administered at least 2 doses of the 3.6 mg goserelin acetate implant. Optionally, after the second dose is administered, subsequent doses of the 3.6 mg goserelin acetate implant are administered to the patient every 28-36 days. In total, 2, 3, 4, 5, 6, 7, 8, 9, 10, 11 and up to 12 doses of the 3.6 mg goserelin acetate implant may be administered. After the second dose of the 3.6 mg goserelin acetate implant or optional subsequent doses of the 3.6 mg goserelin acetate implant, the 3.6 mg goserelin acetate implant is discontinued, and a 10.8 mg goserelin implant is then administered to the patient once every 84 to 108 days. In other words, the patient is switched from the 3.6 mg goserelin acetate implant administered once every 28-36 days to the 10.8 mg goserelin implant administered once every 84 to 108 days.

In aspect ii), after the initial dose of the 3.6 mg goserelin acetate implant, the 3.6 mg goserelin acetate implant is discontinued, and a 10.8 mg goserelin implant is administered to the patient once every 84 to 108 days. In other words, a single dose of 3.6 mg goserelin acetate implant is administered, followed by a switch to the 10.8 mg goserelin implant administered once every 84 to 108 days. One reason for switching may be patient noncompliance, that is, the patient received a single dose of the 3.6 mg goserelin acetate and failed to return for a second dose within 28-36 days.

In the method, administering the 3.6 mg goserelin implant is concomitant with an initial neoadjuvant or adjuvant IV chemotherapy cycle. Subsequent administrations of the 3.6 mg goserelin implant and/or 10.8 mg goserelin acetate implants can be continued during subsequent neoadjuvant or adjuvant IV chemotherapy cycles. That is, goserelin is administered during the course of chemotherapy. In an aspect, the course of chemotherapy is 8 weeks, 12 weeks, 16, weeks, 20 weeks, 6 months, 8 months, 10 months or 12 months. In a further aspect, administering the 10.8 mg goserelin implant to the patient once every 84 to 108 days can be continued after termination of the course of chemotherapy, for example, for the lifetime of the patient. In an aspect, administering the 10.8 mg goserelin implant to the patient once every 84 to 108 days is continued for at least 5 years.

In an aspect, in option i) the patient was noncompliant and did not return 28-36 days after the second dose of the 3.6 mg goserelin acetate implant for a subsequent dose of 3.6 mg goserelin acetate implant. In another aspect, in option i), the patient was noncompliant and did not return 28-36 days after the subsequent dose of the 3.6 mg goserelin acetate implant for a further subsequent dose of 3.6 mg goserelin acetate implant. In an aspect in option ii), the patient was noncompliant and did not return 28-36 days after the initial dose of the 3.6 mg goserelin acetate implant for a second dose of 3.6 mg goserelin acetate implant.

In an aspect, the patient is a woman with early-stage breast cancer. In another aspect, the patient is a woman with advanced breast cancer.

In an aspect, the method further comprises administering to the patient radiation therapy, hormone therapy, targeted cancer therapy, immunotherapy, or a combination thereof.

In another aspect, prior to discontinuing the 3.6 mg goserelin acetate implant, patient tolerance to the 3.6 mg goserelin acetate implant is established. Patient tolerance to the 3.6 mg goserelin acetate implant can be defined as limited untoward effects that preclude subsequent administration such as rash, hypersensitivity, anaphylaxis, and the like. Tolerance does not include the expected effects of hormone suppression such as hot flashes and the like.

### Long-Term Chemotherapy

In an aspect, a method of treating hormone receptor positive breast cancer in a patient comprises administering a plurality of long-term IV chemotherapy cycles to the patient to provide a long-term course of chemotherapy, concomitantly administering an initial dose of a 3.6 mg goserelin acetate implant to the patient when a first of the plurality of long-term IV chemotherapy cycles is administered, and either
i) 28-36 days after the initial dose of the 3.6 mg goserelin acetate implant, administering a second dose of the 3.6 mg goserelin acetate implant to the patient, optionally administering subsequent doses of the 3.6 mg goserelin acetate implant to the patient every 28-36 days, and after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days, or
ii) after the initial dose of the 3.6 mg goserelin acetate implant, discontinuing the 3.6 mg goserelin acetate implant, and administering a 10.8 mg goserelin implant to the patient once every 84 to 108 days,
wherein in i) or ii) administering the 10.8 mg goserelin implant to the patient once every 84 to 108 days is continued for the lifetime of the patient.

In aspect i), 28-36 days after the initial dose of the 3.6 mg goserelin acetate implant is administered, a second dose of the 3.6 mg goserelin acetate implant is administered to the patient. That is, the patient is administered at least 2 doses of the 3.6 mg goserelin acetate implant. Optionally, after the second dose is administered, subsequent doses of the 3.6 mg goserelin acetate implant are administered to the patient every 28-36 days. In total, 2, 3, 4, 5, 6, 7, 8, 9, 10, 11 and up to 12 doses of the 3.6 mg goserelin acetate implant may be administered. After the second dose of the 3.6 mg goserelin acetate implant or optional subsequent doses of the 3.6 mg goserelin acetate implant, the 3.6 mg goserelin acetate implant is discontinued, and a 10.8 mg goserelin implant is then administered to the patient once every 84 to 108 days. In other words, the patient is switched from the 3.6 mg goserelin acetate implant administered once every 28-36 days to the 10.8 mg goserelin implant administered once every 84 to 108 days.

In aspect ii), after the initial dose of the 3.6 mg goserelin acetate implant, the 3.6 mg goserelin acetate implant is discontinued, and a 10.8 mg goserelin implant is administered to the patient once every 84 to 108 days. In other words, a single dose of 3.6 mg goserelin acetate implant is administered, followed by a switch to the 10.8 mg goserelin implant administered once every 84 to 108 days. One reason for switching may be patient noncompliance, that is, the patient received a single dose of the 3.6 mg goserelin acetate and failed to return for a second dose within 28-36 days.

In the method, administering the 3.6 mg goserelin implant is concomitant with a first of the plurality of long-term IV chemotherapy cycles. Subsequent administrations of the 3.6 mg goserelin implant and/or 10.8 mg goserelin acetate implants are continued during subsequent IV chemotherapy cycles. That is, goserelin is administered during a long-term course of chemotherapy. In an aspect, a long-term course of chemotherapy is the lifetime of the patient or at least 5 years. In a further aspect, administering the 10.8 mg goserelin implant to the patient once every 84 to 108 days can be continued after termination of the long-term course of chemotherapy.

In an aspect, in option i) the patient was noncompliant and did not return 28-36 days after the second dose of the 3.6 mg goserelin acetate implant for a subsequent dose of 3.6 mg goserelin acetate implant. In another aspect, in option i), the patient was noncompliant and did not return 28-36 days after the subsequent dose of the 3.6 mg goserelin acetate implant for a further subsequent dose of 3.6 mg goserelin acetate implant. In an aspect in option ii), the patient was noncompliant and did not return 28-36 days after the initial dose of the 3.6 mg goserelin acetate implant for a second dose of 3.6 mg goserelin acetate implant.

In an aspect, the patient is a woman with early-stage breast cancer. In another aspect, the patient is a woman with advanced breast cancer.

In an aspect, the method further comprises administering to the patient radiation therapy, hormone therapy, targeted cancer therapy, immunotherapy, or a combination thereof.

In another aspect, prior to discontinuing the 3.6 mg goserelin acetate implant, patient tolerance to the 3.6 mg goserelin acetate implant is established. Patient tolerance to the 3.6 mg goserelin acetate implant can be defined as limited untoward effects that preclude subsequent administration such as rash, hypersensitivity, anaphylaxis, and the like. Tolerance does not include the expected effects of hormone suppression such as hot flashes and the like.

### Methods of Improving Patient Compliance

In an aspect, the two dosage strengths of goserelin can be leveraged to improve patient compliance, increase duration of treatment, and reduce the need for oophorectomy in a patient with hormone receptor positive breast cancer. Gonadotropin-releasing hormone analogs such as goserelin are advantageous over oophorectomy as ovarian suppression can be reversible, while oophorectomy is not reversible. In addition to ovarian suppression, it is believed that goserelin reduces recurrence of disease (increases disease-free survival). However, the requirement for monthly subcutaneous administration of the 3.6 mg goserelin acetate implant can result in delay of monthly dosing, missed doses, and discontinuation of therapy. Once tolerability of the 3.6 mg goserelin acetate implant is established, it can be advantageous to switch to the 10.8 mg goserelin implant administered once every 84 to 108 days to alleviate some of the disadvantages of once monthly dosing. Dosing once every 3 months will provide less frequent transient injection site or pain, for example, which has been reported in some cases.

Specifically, once a dose of 3.6 mg goserelin acetate implant is administered, a patient is determined to be nonadherent when a second or subsequent dose of 3.6 mg goserelin acetate implant is not administered within 36 days after the previous dose. Switching to the 10.8 mg dosage form will provide less frequent dosing and is expected to improve patient compliance, increase duration of treatment, and reduce the need for oophorectomy.

In an aspect, a method of improving patient compliance, increasing duration of treatment, and/or reducing the need for oophorectomy in a patient with hormone receptor positive breast cancer comprises
i) administering an initial dose of a 3.6 mg goserelin acetate implant to the patient,
administering one or more subsequent doses of the 3.6 mg goserelin acetate implant to the patient, wherein each subsequent dose is prescribed to be administered 4 weeks after a preceding dose,
identifying the patient as nonadherent if the patient does not receive the subsequent dose of 3.6 mg goserelin acetate implant at less than or equal to 36 days after the preceding dose,
discontinuing the 3.6 mg goserelin acetate implant in the nonadherent patient,
administering a 10.8 mg goserelin implant to the nonadherent patient, wherein the 10.8 mg goserelin implant is prescribed to be administered once every 12 weeks, and
continuing to administer the 10.8 mg goserelin implant is administered once every 84 to 108 days to improve patient compliance, increase duration of treatment, and/or reduce the need for oophorectomy,
wherein a course of treatment with a combination of 3.6 mg goserelin acetate implant and 10.8 mg goserelin implant is two years or the lifetime of the patient; or
ii) administering an initial dose of a 3.6 mg goserelin acetate implant to the patient, wherein a subsequent dose is prescribed to be administered 4 weeks after the first dose,
identifying the patient as nonadherent when the patient does not receive the subsequent dose of 3.6 mg goserelin acetate implant at less than or equal to 36 days after the first dose,
discontinuing the 3.6 mg goserelin acetate implant in the nonadherent patient, and
administering a 10.8 mg goserelin implant to the nonadherent patient, wherein the 10.8 mg goserelin implant is prescribed to be administered once every 12 weeks,
wherein the 10.8 mg goserelin implant is administered once every 84 to 108 days for a minimum of eight doses to improve patient compliance, increase duration of treatment, and/or reduce the need for oophorectomy.

In i), in addition to the first dose of 3.6 mg goserelin acetate implant, the patient may receive 1, 2, 3, 4, 5, 6, 7, 8 or more subsequent doses of the 3.6 mg goserelin acetate implant prior to becoming nonadherent. Once the patient becomes nonadherent on the 3.6 mg goserelin acetate implant, the patient is switched to the 10.8 mg goserelin implant.

In an aspect, it may be preferred that the patient is administered 2, 3 or more doses of 3.6 mg goserelin acetate implant before switching to the higher dose 10.8 mg goserelin implant.

In an aspect, the patient is a woman with early-stage breast cancer. In another aspect, the patient is a woman with advanced breast cancer.

In an aspect, the method further comprises administering to the patient radiation therapy, hormone therapy, targeted cancer therapy, immunotherapy, or a combination thereof.

In another aspect, prior to discontinuing the 3.6 mg goserelin acetate implant, patient tolerance to the 3.6 mg goserelin acetate implant is established. Patient tolerance to the 3.6 mg goserelin acetate implant can be defined as limited untoward effects that preclude subsequent administration such as rash, hypersensitivity, anaphylaxis, and the like. Tolerance does not include the expected effects of hormone suppression such as hot flashes and the like.

In this method, rather than the patient being nonadherent, the patient may consider the every 4 weeks dosing to be inconvenient. In this case, the patient may be switched from the 3.6 mg goserelin acetate implant to the 10.8 mg implant to improve convenience.

In addition, switching to the 10.8 mg dosage form can improve health care utilization by reducing the number of clinic visits for the patient.

The invention is further illustrated by the following non-limiting examples.

### EXAMPLES

### Methods

Electronic health record data of adult patients with a history of breast cancer and with ≥2 prescriptions of goserelin between 1 Jan. 2017-31 Dec. 2022 were identified through TriNetX. The index date was set as the initiation of goserelin administration. Patients were followed until 15 Mar. 2024. Patient demographics, treatment adherence, and healthcare resource utilization (HCRU) were examined and summarized using descriptive analytics.

The study eligibility were as follows:

### Inclusion:

Women aged 18 years or older
Two or more prescriptions of goserelin (3.6 mg or 10.8 mg dose) on or after January 1,2017 and prior to Dec. 31, 2022
History of primary breast cancer diagnosis prior to first goserelin prescription.

### Exclusion:

Prescription of goserelin in the database prior to Jan. 1, 2017

The analysis was stratified by goserelin dose group as follows:
1. Patients treated with goserelin 3.6 mg only during the follow-up period
2. Patients treated with goserelin 10.8 mg only during the follow-up period
3. Patients who switched from goserelin 3.6 mg to 10.8 mg at any point during the follow-up period

The results are provided in Table 1:

TABLE 1
Summary of Study Results

| | | | | Switched from | |
|---|---|---|---|---|---|
| | | | | goserelin | |
| | Goserelin | | Goserelin | 3.6 mg to | |
| | 3.6 mg | | 10.8 mg | 10.8 mg | |
| | (n = 2,870) | | (n = 410) | (n = 340) | |
| Median duration of | 264 | | 429 | 776 | |
| treatment (days) | | | | | |
| Median time to switch | — | | — | 218 | |
| from 3.6 mg to 10.8 | | | | | |
| mg (days) | | | | | |
| Patients adherent* to | 1,608 | (56.4%) | 306 (74.4%) | — | |
| recommended dosing | | | | | |
| schedule | | | | | |
| Status as of Mar. | | | | | |
| 15, 2024 | | | | | |
| Discontinued goserelin | 2,018 | (70.8%) | 261 (64.3%) | 173 | (50.9%) |
| Death | 88 | (3.1%) | 10 (2.5%) | 3 | (0.9%) |

*Patients are adherent if: ≤36 days between prescriptions (3.6 mg) or ≤108 days between prescriptions (10.8 mg)

Overall, 3,620 patients were identified: 2,870 treated with goserelin 3.6 mg, 410 with goserelin 10.8 mg, and 340 who switched from goserelin 3.6 mg to 10.8 mg. Peak utilization of goserelin 10.8 mg (36.6%) and peak dosage switching (26.5%) occurred in 2020. Across groups, mean age at index date was 42.2-44.4 years, and patients were white (64.1-67.6%), black (11.8%- 13.9%), Asian (8.0%- 11.8%), and American Indian and/or Alaskan native (0.3%- 2.9%). Of the patients with known BMI, BMI mostly ranged between 18.5-24.9 kg/m2 in all groups. Patients who switched from goserelin 3.6 mg to 10.8 mg had the longest median treatment duration (776 days) (Table). Patients treated with goserelin 10.8 mg remained on treatment for a median of 426 days, and 74.4% of these patients were treatment-adherent (Table 1). In comparison, patients treated with goserelin 3.6 mg had a shorter median treatment duration (226 days) and were less adherent (56.4%) (Table). Healthcare resource utilization (HCRU) during the 12 months after index date was broadly similar across groups.

Treatment with goserelin 10.8 mg every three months is associated with greater adherence and longer duration of treatment, compared with 3.6 mg once monthly, in patients with a history of breast cancer. Patients who switched from goserelin 3.6 mg to goserelin 10.8 mg were treatment-adherent for nearly two years, consistent with treatment recommendations.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of treating hormone receptor positive breast cancer in a patient, comprising
concomitantly administering an initial neoadjuvant or adjuvant IV chemotherapy cycle and an initial dose of a 3.6 mg goserelin acetate implant to the patient, optionally administering subsequent neoadjuvant or adjuvant IV chemotherapy cycles to the patient, wherein the subsequent neoadjuvant or adjuvant IV chemotherapy cycles are the same or different from the initial neoadjuvant or adjuvant IV chemotherapy cycle and each other, and either
i) 28-36 days after the initial dose of the 3.6 mg goserelin acetate implant, administering a second dose of the 3.6 mg goserelin acetate implant to the patient,
optionally administering subsequent doses of the 3.6 mg goserelin acetate implant to the patient every 28-36 days, and
after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days,
or
ii) after the initial dose of the 3.6 mg goserelin acetate implant, discontinuing the 3.6 mg goserelin acetate implant, and administering a 10.8 mg goserelin implant to the patient once every 84 to 108 days.

2. The method of claim 1, wherein i) or ii) further comprises administering the 10.8 mg goserelin implant to the patient once every 84 to 108 days throughout a course of neoadjuvant or adjuvant IV chemotherapy.

3. The method of claim 2, wherein the course of chemotherapy is 8 months to 12 months.

4. The method of claim 1, wherein in i) or ii), administering the 10.8 mg goserelin implant to the patient once every 84 to 108 days is continued for the lifetime of the patient.

5. The method of claim 1, wherein in i) or ii), wherein administering the 10.8 mg goserelin implant to the patient once every 84 to 108 days is continued for at least 5 years.

6. The method of claim 1, wherein 1 to 12 subsequent neoadjuvant or adjuvant IV chemotherapy cycles are administered to the patient.

7. The method of claim 1, wherein in option i), the patient was noncompliant and did not return 28-36 days after the second dose of the 3.6 mg goserelin acetate implant for a subsequent dose of 3.6 mg goserelin acetate implant.

8. The method of claim 1, wherein in option i), the patient was noncompliant and did not return 28-36 days after the subsequent dose of the 3.6 mg goserelin acetate implant for a further subsequent dose of 3.6 mg goserelin acetate implant.

9. The method of claim 1, wherein in option ii), the patient was noncompliant and did not return 28-36 days after the initial dose of the 3.6 mg goserelin acetate implant for a second dose of 3.6 mg goserelin acetate implant.

10. The method of claim 1, wherein the patient is a woman with early-stage breast cancer.

11. The method of claim 1, wherein the patient is a woman with advanced breast cancer.

12. The method of claim 1, further comprising administering to the patient radiation therapy, hormone therapy, targeted cancer therapy, immunotherapy, or a combination thereof.

13. The method of claim 1, wherein prior to discontinuing the 3.6 mg goserelin acetate implant, patient tolerance to the 3.6 mg goserelin acetate implant is established.

14. A method of treating hormone receptor positive breast cancer in a patient, comprising
administering a plurality of long-term IV chemotherapy cycles to the patient to provide a long-term course of chemotherapy,
concomitantly administering an initial dose of a 3.6 mg goserelin acetate implant to the patient when a first of the plurality of long-term IV chemotherapy cycles is administered, and either
i) 28-36 days after the initial dose of the 3.6 mg goserelin acetate implant, administering a second dose of the 3.6 mg goserelin acetate implant to the patient,
optionally administering subsequent doses of the 3.6 mg goserelin acetate implant to the patient every 28-36 days, and
after the second dose of the 3.6 mg goserelin acetate implant or the optional subsequent doses of the 3.6 mg goserelin acetate implant, discontinuing the 3.6 mg goserelin acetate implant, and administering a 10.8 mg goserelin implant to the patient once every 84 to 108 days,
or
ii) after the initial dose of the 3.6 mg goserelin acetate implant, discontinuing the 3.6 mg goserelin acetate implant, and administering a 10.8 mg goserelin implant to the patient once every 84 to 108 days,
wherein in i) or ii) administering the 10.8 mg goserelin implant to the patient once every 84 to 108 days is continued for the lifetime of the patient.

15. The method of claim 14, wherein the long-term course of chemotherapy is the lifetime of the patient.

16. The method of claim 14, wherein the long-term course of chemotherapy is at least 5 years.

17. The method of claim 14, wherein in option i), the patient was noncompliant and did not return 28-36 days after the second dose of the 3.6 mg goserelin acetate implant for a subsequent dose of 3.6 mg goserelin acetate implant.

18. The method of claim 14, wherein in option i), the patient was noncompliant and did not return 28-36 days after the subsequent dose of the 3.6 mg goserelin acetate implant for a further subsequent dose of 3.6 mg goserelin acetate implant.

19. The method of claim 14, wherein in option ii), the patient was noncompliant and did not return 28-36 days after the initial dose of the 3.6 mg goserelin acetate implant for a second dose of 3.6 mg goserelin acetate implant.

20. The method of claim 14, wherein the patient is a woman with early-stage breast cancer.

21. The method of claim 14, wherein the patient is a woman with advanced breast cancer.

22. The method of claim 14, further comprising administering to the patient radiation therapy, hormone therapy, targeted cancer therapy, immunotherapy, or a combination thereof.

23. The method of claim 14, wherein prior to discontinuing the 3.6 mg goserelin acetate implant, patient tolerance to the 3.6 mg goserelin acetate implant is established.

24. A method of improving patient compliance, increasing duration of treatment, and/or reducing the need for oophorectomy in a patient with hormone receptor positive breast cancer comprises
i) administering an initial dose of a 3.6 mg goserelin acetate implant to the patient,
administering one or more subsequent doses of the 3.6 mg goserelin acetate implant to the patient, wherein each subsequent dose is prescribed to be administered 4 weeks after a preceding dose,
identifying the patient as nonadherent if the patient does not receive the subsequent dose of 3.6 mg goserelin acetate implant at less than or equal to 36 days after the preceding dose,
discontinuing the 3.6 mg goserelin acetate implant in the nonadherent patient,
administering a 10.8 mg goserelin implant to the nonadherent patient, wherein the 10.8 mg goserelin implant is prescribed to be administered once every 12 weeks, and
continuing to administer the 10.8 mg goserelin implant is administered once every 84 to 108 days to improve patient compliance, increase duration of treatment, and/or reduce the need for oophorectomy,
wherein a course of treatment with a combination of 3.6 mg goserelin acetate implant and 10.8 mg goserelin implant is two years or the lifetime of the patient;
or
ii) administering an initial dose of a 3.6 mg goserelin acetate implant to the patient, wherein a subsequent dose is prescribed to be administered 4 weeks after the first dose,
identifying the patient as nonadherent when the patient does not receive the subsequent dose of 3.6 mg goserelin acetate implant at less than or equal to 36 days after the first dose,
discontinuing the 3.6 mg goserelin acetate implant in the nonadherent patient, and
administering a 10.8 mg goserelin implant to the nonadherent patient, wherein the 10.8 mg goserelin implant is prescribed to be administered once every 12 weeks,
wherein the 10.8 mg goserelin implant is administered once every 84 to 108 days for a minimum of eight doses to improve patient compliance, increase duration of treatment and/or reduce the need for oophorectomy.

25. The method of claim 24, wherein in i), the patient is administered 2 or 3 subsequent doses of the 3.6 mg goserelin acetate implant.

26. The method of claim 24, wherein the patient is a woman with early-stage breast cancer.

27. The method of claim 24, wherein the patient is a woman with advanced breast cancer.

28. The method of claim 24, further comprising administering to the patient radiation therapy, hormone therapy, targeted cancer therapy, immunotherapy, or a combination thereof.

29. The method of claim 24, wherein prior to discontinuing the 3.6 mg goserelin acetate implant, patient tolerance to the 3.6 mg goserelin acetate implant is established.
